(19) United States
(12) Patent Application Publication
(10) Pub. No.: US 2022/0127562 A1
(43) Pub. Date: Apr. 28, 2022

(54) COMPOSITION FOR DEGRADATION OF MYCOTOXIN COMPRISING ASPERGILLUS CULTURE FILTRATE CONTAINING IRON COMPOUND OR YEAST EXTRACT AS EFFECTIVE COMPONENT AND USES THEREOF

(71) Applicant: SKYANGELBIO Corp., Seoul (KR)

(72) Inventors: Jaehyuk YU, Madison, WI (US); Dasol CHOI, Madison, WI (US); Ahmad Fakhri Mohammad ALSHANNAQ, Madison, WI (US)

(21) Appl. No.: 17/081,226

(22) Filed: Oct. 27, 2020

(51) Int. Cl. C12N 1/16; A23L 5/20; A23K 10/12; A23K 10/18

(52) U.S. Cl. CPC C12N 1/16; A23L 5/28; A23V 2002/00; A23K 10/12; A23K 10/18; A23L 5/276

(57) ABSTRACT

A composition for degradation of mycotoxin including, as an effective component, Aspergillus culture filtrate which contains an iron compound or yeast extract, and uses thereof, are proposed. As aflatoxin can be degraded with high efficiency and simple composition by the composition of the present invention and the activity of degrading mycotoxin is maintained in very stable state in a broad temperature range of from room temperature to heating at 121° C. for sterilization, it is expected that the composition can be advantageously used for processing like heating at high temperatures.

### TECHNICAL FIELD

The present invention relates to compositions for degradation of mycotoxin comprising, as an effective component, Aspergillus culture filtrate which contains an iron compound and/or yeast extract, and uses thereof.

### BACKGROUND ART

Aflatoxins (AFs) are one type of the toxins of toxic molds, and they are difuranocoumarin derivatives produced by Aspergillus flavus, Aspergillus parasiticus, Aspergillus nomius, or the like of genus Aspergillus via polyketide pathway. Aflatoxins are detected from various kinds of agricultural food products, and are known to be a causal agent of the turkey X disease in 1960s. At present moment, about 20 types of aflatoxins are known, and B1, M1, B2, G1, and G2, which are the most important types among them, are widely found in nature. Aflatoxins are classified into Group 1 human carcinogens, and aflatoxin B1 is most commonly found and classified as the most acute and potent toxic carcinogen. After being activated by cytochrome P450 in human liver, aflatoxin B1 converts into aflatoxin B1-8,9-oxide, which binds to DNA of p53 for causing cancer. It is also known that aflatoxins cause a disturbance in reproduction-related hormones in cattle to yield sterility, miscarriage, or the like. As such, in many countries, strict management of aflatoxins is effected along with the regulations imposed on the maximum permissible levels of aflatoxins in foods and animal feeds.

As a method for reducing aflatoxins, there are methods of chemical reduction, physical reduction, and biological reduction. For the crop production stage, storage stage after harvest, and storage stage after processing, the chemical reduction method has been mainly proposed while the physical reduction method is carried out mainly at the storage stage after harvest and also at the processing stage. However, once food products are contaminated with aflatoxins, it is very difficult to remove the toxins, and, as the toxins are not degraded even by heating, development of a technique for effective removal of aflatoxins is required.

Meanwhile, in Korean Patent Registration No. 2005433, “Streptomyces panaciradicis AF34 strain for degradation of aflatoxin” is described, and, in Korean Patent Registration No. 0380535, “Method for control of aflatoxin using inhibitory microorganism CP220 and bean fermentation food and animal feed utilizing such microorganism” is described. However, there is no description included in those documents regarding the “composition for degradation of mycotoxin comprising, as an effective component, Aspergillus culture filtrate which contains an iron compound or yeast extract and uses thereof” of the present invention.

### DETAILED DESCRIPTION OF THE INVENTION

### Technical Problems to be Solved

The present invention is devised under the circumstances described above, and the inventors of the present invention found that the culture filtrate of a strain belonging to genus Aspergillus has an excellent degrading activity for aflatoxins and patulin, and, as a result of analyzing the degrading activity for aflatoxins or patulin after preparing various culture filtrates by modifying the composition of a culture medium to optimize the degrading activity, the inventors found that an iron component or yeast extract is required for having the degrading activity for aflatoxins and patulin. It was additionally found that, as a result of controlling the content of a compound which has an iron element and yeast extract, the culture filtrate of a strain belonging to genus Aspergillus of the present invention can have enhanced degrading activity for aflatoxins and patulin, and the culture filtrate can exhibit the degrading activity for aflatoxins over a broad temperature range and, in particular, have enhanced degrading activity for aflatoxins at high temperature conditions, and the present invention is completed accordingly.

### Technical Means for Solving Problems

To solve the problems described in the above, the present invention provides a composition for degradation of mycotoxin comprising, as an effective component, an iron compound; yeast extract; or culture filtrate of Aspergillus cultured in culture medium containing one or more of an iron component and yeast extract.

The present invention further provides a method for degradation of mycotoxin including treating a sample suspected to contain mycotoxin with the aforementioned composition.

The present invention further provides a method for production of Aspergillus culture filtrate having an activity of degrading mycotoxin including: (a) inoculating Aspergillus conidia to a culture medium containing one or more of an iron compound and yeast extract followed by culturing; and (b) filtering a culture broth of the Aspergillus of above (a), and also provides an Aspergillus culture filtrate having an activity of degrading mycotoxin that is produced by the aforementioned method.

The present invention further provides a food additive comprising the aforementioned composition for degradation of mycotoxin of the present invention.

The present invention still further provides an animal feed additive comprising the aforementioned composition for degradation of mycotoxin of the present invention.

### Advantageous Effect of the Invention

Compared to techniques of a related art, the composition for degradation of mycotoxin according to the present invention can degrade aflatoxins with high efficiency and simple composition, and, as the activity of degrading mycotoxin is maintained in very stable state in a broad temperature range from room temperature to heating at 121° C. for sterilization, it is expected that the composition of the present invention can be advantageously used for processing like treatment at high temperatures. Accordingly, it is expected that the composition of the present invention can be advantageously used, in the field of food products and animal feeds for which biodegradation of mycotoxin (in particular, aflatoxins or patulin) is required, as a novel material that can maintain the activity of degrading mycotoxin even at high temperatures.

### BRIEF DESCRIPTION OF THE DRAWINGS

FIG. 1 shows the process of producing D-Tox B, D-Tox BR, and D-Tox YR of the present invention.

FIG. 2 shows the result of determining the aflatoxin B1 degrading activity of a mixture solution of iron sulfate and zinc sulfate over a broad concentration range.

FIGS. 3a-3b show the result of determining the aflatoxin B1 degrading activity of an iron sulfate solution and a zinc sulfate solution.

FIG. 4 shows the result of determining the degrading activity of 2.5% or 5% iron sulfate solution for 1,000 ppb aflatoxin B1 at room temperature.

FIG. 5 shows the result of determining the degrading activity of an iron sulfate solution for 1,000 ppb aflatoxin B1, in which the determination was made based on organic solvent extraction method using chloroform.

FIG. 6 shows the result of determining the degrading activity of a solution of various compounds containing iron component (concentration: 4%) for 1,000 ppb aflatoxin B1.

FIG. 7 shows the result of determining the degrading activity of D-Tox B and D-Tox B^{0.5}, in which content of D-glucose and 20× sodium nitrate solution has been cut down by half, for aflatoxin B1 at high temperature (100° C.) condition.

FIGS. 8a-8b show the result of determining the degrading activity of D-Tox B_{0.1}^{0.5} R50, in which iron sulfate has been additionally added and zinc sulfate has been reduced (see Table 3), for 1,000 or 500 ppb aflatoxin B1.

FIG. 9 shows the result of determining the degrading activity of D-Tox B₁^{0.5} R5 and D-Tox B₁^{0.5} R10, in which iron sulfate and zinc sulfate have been optimized (see Table 3), for 500 ppb aflatoxin B1.

FIGS. 10a-10b show the result of determining the degrading activity of D-Tox B₁^{0.5} R10 for 50 or 500 ppb aflatoxin B1 at room temperature.

FIGS. 11a-11b show the result of determining the degrading activity of D-Tox B₁^{0.5} R10 for 500 ppb aflatoxin B1 after various treatments.

FIG. 12 shows the result of determining the degrading activity of D-Tox B₁^{0.5} R5, in which yeast extract has been additionally added, for aflatoxin B1.

FIG. 13 shows the result of determining D-Tox activation of D-Tox Y_{0.1}^{0.5} R5 in which yeast extract has been added (see Table 3).

FIGS. 14a-14b show the result of determining the degrading activity of D-Tox Y₁^{0.5} R10 and D-Tox Y₅^{0.5} R10 (see Table 3) for aflatoxin B1 depending on concentration of yeast extract.

FIGS. 15a-15b show the result of determining the degrading activity of D-Tox Y₅^{0.5} R10 and D-Tox Y₅⁰ R10 (see Table 3) for aflatoxin B1 at different concentrations depending on use amount of glucose and sodium nitrate.

FIG. 16 shows the result of determining the degrading activity of D-Tox Y₅⁰ R0, D-Tox Y₅⁰ R10, D-Tox Y₅⁰ R20, D-Tox Y₅⁰ R40 and D-Tox Y₅⁰ R80 (see Table 3) for aflatoxin B1 depending on composition with yeast extract (5 g per liter) and iron sulfate at various concentrations (0 to 80 ppm)

FIGS. 17a-17b show the result of determining the degrading activity of D-Tox B₁^{0.5} R5 and D-Tox B₁^{0.5} R10 (see Table 3) for 1,000 ppb patulin.

FIG. 18 shows the result of determining D-Tox activation of D-Tox YR, in which yeast extract has been added (see Table 3), for 1,000 ppb patulin.

FIG. 19 shows the result of determining the degrading activity of D-Tox Y₅⁰ R10 (see Table 3) and control group for patulin.

### BEST MODE(S) FOR CARRYING OUT THE INVENTION

To achieve the object of the present invention, the present invention provides a composition for degradation of mycotoxin comprising, as an effective component, (a) an iron compound; (b) yeast extract; or (c) culture filtrate of Aspergillus cultured in culture medium containing one or more of an iron component and yeast extract.

With regard to the composition for degradation of mycotoxin according to the present invention, the iron compound may be an iron salt, and the ion salt may be ferrous sulfate heptahydrate, ferric sulfate, ferric chloride anhydrous, ferric chloride hexahydrate, ferrous chloride tetrahydrate, ferric nitrate, or ferrous ammonium sulfate, but it is not limited thereto.

Furthermore, with regard to the composition for degradation of mycotoxin according to the present invention, the culture medium may contain only one of an iron compound and yeast extract, or it may contain both the iron compound and yeast extract.

As described herein, the term “culture filtrate” means a liquid material that is obtained by growing an organism (in particular, mold or bacteria) in a liquid medium and filtering the resultant through a filter paper, and included in the culture filtrate are various enzymes, proteins, sugars, amino acids, antibiotics, or the like which have been discharged into the liquid medium during the process of growing an organism.

With regard to the composition for degradation of mycotoxin according to the present invention, the Aspergillus strain can be Aspergillus oryzae (A. oryzae), Aspergillus terreus (A. terreus), Aspergillus sojae (A. sojae), Aspergillus nidulans (A. nidulans), Aspergillus fumigatus (A. fumigatus), or Aspergillus flavus (A. flavus), but it is not limited thereto. The culture filtrate of those strains is characterized by its remarkably excellent activity of degrading mycotoxin compared to a culture filtrate derived from other Aspergillus species.

Furthermore, with regard to the composition for degradation of mycotoxin according to the present invention, the mycotoxin can be aflatoxin, patulin, ochratoxin, fumonisin, zearalenone, deoxynivalenol, or trichothecene, preferably aflatoxin, patulin, or ochratoxin, and most preferably aflatoxin or patulin, but it is not limited thereto.

Furthermore, as the composition for degradation of mycotoxin according to the present invention is characterized in that it exhibits the activity of degrading mycotoxin in a temperature range of 20 to 150° C., and preferably in a temperature range of 20 to 130° C., it can be applied in a broad temperature range. In addition, the function of the composition can be stably exhibited even during a heat treatment process with temperature of more than 100° C., and thus it can be industrially used with very high usefulness.

With regard to the composition for degradation of mycotoxin according to one embodiment of the present invention, the Aspergillus culture filtrate can be a sterile cell-free culture broth which is obtained by inoculating, to 150-1,000 ml culture medium, Aspergillus oryzae conidia to have conidia concentration of 10⁴ to 10⁷ conidia/ml of culture medium, carrying out culture for 6 to 10 days at 30° C. under shaking at 150-220 rpm, removing mycelia from the culture broth, and filtering the resultant using a filter unit for sterilization, but it is not limited thereto.

The present invention further provides a method for degradation of mycotoxin including treating a sample suspected to contain mycotoxin with the composition of the present invention.

With regard to the method for degradation of mycotoxin according to the present invention, the composition comprises, as an effective component, Aspergillus culture filtrate which has an activity of degrading mycotoxin, and the details of the culture filtrate are the same as those described in the above.

Furthermore, with regard to the method for degradation of mycotoxin according to the present invention, the mycotoxin is the same as those described in the above, and the sample suspected to contain mycotoxin can be an agricultural product, a processed food product, or an animal feed, but it is not limited thereto.

The present invention further provides a method for production of Aspergillus culture filtrate having an activity of degrading mycotoxin including: (a) inoculating Aspergillus conidia to a culture medium containing one or more of an iron compound and yeast extract followed by culturing; and (b) filtering a culture broth of the Aspergillus of above (a), and also provides an Aspergillus culture filtrate having an activity of degrading mycotoxin that is produced by the aforementioned method.

Furthermore, with regard to the production method according to the present invention, the Aspergillus of above (a) can be A. oryzae, A. terreus, A. sojae, A. nidulans, A. fumigatus, or A. flavus, but it is not limited thereto.

Furthermore, the conidia of above (a) can be inoculated to a culture medium containing one or more of an iron compound and yeast extract to have a concentration of from 10⁴ to 10⁷ conidia/ml, and preferably can be inoculated to a culture medium to have concentration of 5×10⁵ conidia/ml, and cultured for 6 to 10 days under shaking at 150 rpm, 30° C., but it is not limited thereto.

With regard to the production method according to one embodiment of the present invention, the culture medium can be a medium which contains only one of an iron compound and yeast extract or both the iron compound and yeast extract. The iron compound may be, although not limited thereto, an iron salt such as ferrous sulfate heptahydrate, ferric sulfate, ferric chloride anhydrous, ferric chloride hexahydrate, ferrous chloride tetrahydrate, ferric nitrate, or ferrous ammonium sulfate, and preferably ferrous sulfate heptahydrate (FeSO₄.7H₂0), but it is not limited thereto.

With regard to the production method according to one embodiment of the present invention, the culture medium may specifically contain, per 1 liter unit volume of the culture medium, ferrous sulfate heptahydrate in an amount of 0.1-300 mg and yeast extract in an amount of 0.01-10 g, more specifically contain ferrous sulfate heptahydrate in an amount of 3-12 mg and yeast extract in an amount of 0.05-7 g, and even more specifically contain ferrous sulfate heptahydrate in an amount of 5-10 mg and yeast extract in an amount of 0.1-5 g per 1 liter unit volume of the culture medium, but it is not limited thereto.

Furthermore, in the culture filtrate, glucose, sodium nitrate, magnesium sulfate.heptahydrate, potassium chloride, and potassium phosphate may be additionally contained other than the iron compound and yeast extract. However, it is not always necessary to contain those components. Glucose, sodium nitrate, magnesium sulfate.heptahydrate, potassium chloride, and potassium phosphate may be contained, although not limited thereto, in the following amount: 1-20 g of glucose, 1-10 g of sodium nitrate, 0.1-1.0 g of magnesium sulfate.heptahydrate, 0.1-1.0 g of potassium chloride, and 0.1-2.0 g of potassium phosphate per 1 liter unit volume of the culture medium.

The present invention further provides a food additive comprising the composition for degradation of mycotoxin according to the present invention. When the composition for degradation of mycotoxin according to the present invention is used as a food additive, the composition for degradation of mycotoxin may be either directly added or used in combination with other food ingredients, and it can be suitably used according to a common method. The blending amount of the effective component can be suitably set depending on the purpose of use thereof.

Type of the food product is not particularly limited. Examples of a food product to which the aforementioned material may be added include grains, peanut, pistachio, almond, cotton seed, fruits, meat products, sausages, bread, chocolate, candies, snacks, cookies, pizza, ramen, other noodles, gums, dairy products including ice cream, various soups, beverages, tea, drinks, alcohol beverages and vitamin complexes, and all food products in general sense are included therein.

The present invention still further provides an animal feed additive comprising the composition for degradation of mycotoxin according to the present invention.

As the composition for degradation of mycotoxin according to the present invention comprises Aspergillus culture filtrate having an excellent property of degrading aflatoxin and patulin as mycotoxin, it enables good health state and improved bodyweight gain amount of livestock, and thus the composition can be advantageously used as an effective component of an animal feed additive.

The animal feed additive of the present invention and an animal feed comprising the same may be used with, as an auxiliary component, a material like amino acids, inorganic salts, vitamins, antibiotics, antimicrobial substances, antioxidizing, antimold enzymes, agents for improving digestion and absorption, growth promoting agents, or agents for preventing diseases.

The animal feed additive may be administered to an animal either singly or in combination with other animal feed additives in edible carrier. Furthermore, the animal feed additive can be applied as a top dressing or directly blended in an animal feed. Alternatively, separate from an animal feed, it can be easily administered, in the form of separate oral formulation, by injection or transdermal administration in combination with other components. In general, single daily dosage or divided daily dosage may be taken as it is well known in the pertinent art. When the animal feed additive is administered separately from an animal feed, the administration form of an extract can be prepared, according to combination with non-toxic pharmaceutically acceptable edible carrier, in an immediate-release formulation or a delayed-release formulation, as it is well known in the pertinent art. Examples of the edible carrier include solids and liquids such as corn starch, lactose, sucrose, bean flake, peanut oil, olive oil, sesame oil, or propylene glycol. In case of using a solid carrier, the administration form of an extract can be a tablet, a capsule, a powder, a troche, or a sugar-containing tablet, or top dressing in non-dispersion form. In case of using a liquid carrier, it may have administration form like soft gelatin capsule, syrup, liquid suspension, emulsion, or solution. Furthermore, the administration form may also include an aid such as preservative, stabilizer, wetting agent, emulsifier, or dissolution promoter.

The term “D-Tox” used herein means a cell-free culture filtrate of Aspergillus strain grown in food-grade medium containing human-safe edible chemicals (glucose, nitrates, minerals, cofactors, yeast extract, or the like), and thus it represents a composition showing an excellent activity of degrading aflatoxin as mycotoxin. Characteristics of D-Tox according to the present invention are as described in the following Table 1.

TABLE 1
Characteristics of D-Tox

| Specifications | D-Tox | Other technologies |
|---|---|---|
| Percentage of reduction % | 90-100% | Up to 70-85% |
| Heat and processing stability | Stable | not Stable |
| Aflatoxin degradation ability | Up to 100 ppm | 0.1-5 ppm |
| Protein/non-protein based | Non-protein based | Protein based |
| Single/multiple usability | Multiple | Single |
| Time required for | Short e.g., 20 min | Long e.g., days |
| degradation/removal | | |
| Types of AF detoxification | Irreversible, | Reversible, or binding |
| reactions | destructed | |
| Manufacturing scale-up | Simple, cost- | Not easy, expensive |
| | effective | |
| Recyclable/Environmentally | Yes | No |
| friendly product | | |

Hereinbelow, the present invention is explained in greater detail in view of the Examples. However, it is evident that the following Examples are given only for exemplification of the present invention and by no means the present invention is limited to the following Examples.

### Materials and Methods

### 1. Culture of Aspergillus Strains

Various Aspergillus strains have been used for determining the ability of producing D-Tox (i.e., cell-free culture fermentate with aflatoxin degrading activity), and all the strains were cultured (30° C.) and maintained (4° C.) on potato dextrose agar (PDA) medium (containing 4 g potato starch, 20 g glucose, and 15 g agar in 1 L of distilled water). To prepare inoculum, Aspergillus was grown on PDA for 7 days at 30° C. After that, asexual spores (i.e., conidia) were harvested from the PDA medium by using sterile 0.1% Tween-80 solution. The conidia were counted by using a hemocytometer and adjusted to 10⁸ conidia/ml with sterile distilled water. Conidia suspension was stored at 4° C. and used within 2 weeks after the preparation.

### 2. Composition of Medium for Producing D-Tox

For preparing the full-strength culture medium, 10.0 g D-glucose, 50 ml of 20× sodium nitrate solution, and 1.0 ml of 1,000× zinc sulfate and iron sulfate solution were admixed with one another and dissolved in 600 ml distilled water. After adjusting to the final volume of 1,000 ml, stirring was carried out at least for 20 minutes, and then pH was adjusted to pH 6.5 using hydrochloric acid. Then, according to sterilization under high pressure (50 psi for 20 minutes at 121° C.), the full-strength culture medium was prepared. The 20× sodium nitrate solution and 1,000× zinc sulfate and iron sulfate solution that are used for preparing the medium were prepared as described in the following Table 2.

TABLE 2
Composition of sodium nitrate solution and zinc sulfate
and iron sulfate solution
20× Sodium nitrate solution (based on 1 L)

| | |
|---|---|
| Sodium nitrate (NaNO₃) | 120.0 g |
| Magnesium sulfate•heptahydrate | 10.4 g |
| (MgSO₄•7H₂O) | |
| Potassium chloride (KCl) | 10.4 g |
| Potassium phosphate (KH₂PO₄) | 30.4 g |

1000× Zinc sulfate and iron sulfate solution (based on 1 L)

| | |
|---|---|
| Zinc sulfate•heptahydrate (ZnSO₄•7H₂O) | 22.0 g |
| Ferrous sulfate•heptahydrate (FeSO₄•7H₂O) | 5.0 g |

Furthermore, composition of a culture medium for producing D-Tox in which compositions of glucose, sodium nitrate, zinc sulfate and iron sulfate in culture medium are modified and type of D-Tox according to those compositions are as described in the following Table 3.

TABLE 3
Type of D-Tox with various compositions

| Type of | Name of | Composition of culture medium | |
|---|---|---|---|
| D-Tox | D-Tox | (based on 1 L) | Notes |
| D-Tox | D-Tox | 10.0 g D-glucose, | |
| B | B | 50.0 ml 20× sodium nitrate solution, | |
| | | 1.0 ml zinc sulfate and iron sulfate | |
| | | solution | |
| | | [zinc sulfate heptahydrate 22.0 ppm, | |
| | | ferrous sulfate heptahydrate 5 ppm | |
| | | (final)] | |
| | D-Tox | 5.0 g D-glucose, | Glucose and |
| | B^{0.5} | 25.0 ml 20× sodium nitrate solution, | sodium nitrate |
| | | 1.0 ml zinc sulfate and iron sulfate | are half (½) |
| | | solution | of D-Tox B |
| | | [zinc sulfate heptahydrate 22.0 ppm, | composition |
| | | ferrous sulfate heptahydrate 5 ppm | |
| | | (final)] | |
| D-Tox | D-Tox | 5.0 g D-glucose, | |
| BR | B_{0.1}^{0.5} | 25.0 ml 20× sodium nitrate solution, | |
| | R50 | zinc sulfate heptahydrate 0.1 ppm, | |
| | | ferrous sulfate heptahydrate 50 ppm | |
| | | (final) | |
| | D-Tox | 5.0 g D-glucose, | |
| | B₁^{0.5} R5 | 25.0 ml 20× sodium nitrate solution, | |
| | | zinc sulfate heptahydrate 1 ppm, | |
| | | ferrous sulfate heptahydrate 5 ppm | |
| | | (final) | |
| | D-Tox | 5.0 g D-glucose, | Ferrous sulfate |
| | B₁^{0.5} | 25.0 ml 20× sodium nitrate solution, | heptahydrate |
| | R10 | zinc sulfate heptahydrate 1 ppm, | is added at 2× |
| | | ferrous sulfate heptahydrate 10 ppm | concentration |
| | | (final) | than D-Tox |
| | | | B₁^{0.5} R5 |
| D-Tox | D-Tox | 5.0 g D-glucose, | |
| YR | Y_{0.1}^{0.5} | 25.0 ml 20× sodium nitrate solution, | |
| | R5 | 0.1 g yeast extract + | |
| | | ferrous sulfate heptahydrate 5 ppm | |
| | | (final) | |
| | D-Tox | 5.0 g D-glucose, | |
| | Y₁^{0.5} | 25.0 ml 20× sodium nitrate solution, | |
| | R10 | 1 g yeast extract + | |
| | | ferrous sulfate heptahydrate 10 ppm | |
| | | (final) | |
| | D-Tox | 5.0 g D-glucose, | Yeast extract |
| | Y₅^{0.5} | 25.0 ml 20× sodium nitrate | is added at 5× |
| | R10 | solution, 5 g yeast extract + | concentration |
| | | ferrous sulfate heptahydrate 10 ppm | than D-Tox |
| | | (final) | Y₁^{0.5} R10 |
| | D-Tox | 5 g yeast extract + | Iron |
| | Y₅⁰ R0 | ferrous sulfate heptahydrate 0 ppm | compound, |
| | | (final) | glucose and |
| | | | sodium nitrate |
| | | | are not |
| | | | additionally |
| | | | added |
| | D-Tox | 5 g yeast extract + | Glucose and |
| | Y₅⁰ R10 | ferrous sulfate heptahydrate 10 ppm | sodium nitrate |
| | | (final) | are not |
| | | | additionally |
| | | | added |
| | D-Tox | 5 g yeast extract + | Glucose and |
| | Y₅⁰ R20 | ferrous sulfate heptahydrate 20 ppm | sodium nitrate |
| | | (final) | are not |
| | | | additionally |
| | | | added |
| | D-Tox | 5 g yeast extract + | Glucose and |
| | Y₅⁰ R40 | ferrous sulfate heptahydrate 40 ppm | sodium nitrate |
| | | (final) | are not |
| | | | additionally |
| | | | added |
| | D-Tox | 5 g yeast extract + | Glucose and |
| | Y₅⁰ R80 | ferrous sulfate heptahydrate 80 ppm | sodium nitrate |
| | | (final) | are not |
| | | | additionally |
| | | | added |

* Control: Relevant D-Tox culture medium treated similarly, without fungal inoculation.
* For iron sulfate solution, control is distilled water adjusted as pH 3 with 6N HCl based on the pH of experimental group (iron sulfate solutions).

Summary of the composition of culture medium for preparing D-Tox B, D-Tox BR, and D-Tox YR, which are used in the present invention, based on the description of the above Table 3 is as shown in the following Tables 4 to 6.

TABLE 4
Composition of D-Tox B
and D-Tox BR culture medium (based on 1 L)

| | D-Tox | D-Tox | D-Tox | D-Tox | D-Tox |
|---|---|---|---|---|---|
| | B | B^{0.5} | B_{0.1}^{0.5} R50 | B₁^{0.5} R5 | B₁^{0.5} R10 |
| Glucose | 10 g | 5 g | 5 g | 5 g | 5 g |
| Sodium nitrate | 6 g | 3 g | 3 g | 3 g | 3 g |
| Magnesium | 0.5 g | 0.5 g | 0.5 g | 0.5 g | 0.5 g |
| sulfate | | | | | |
| heptahydrate | | | | | |
| Potassium | 0.5 g | 0.25 g | 0.25 g | 0.25 g | 0.25 g |
| chloride | | | | | |
| Potassium | 1.5 g | 0.75 g | 0.75 g | 0.75 g | 0.75 g |
| phosphate | | | | | |
| Zinc sulfate | 22 mg | 22 mg | 0.1 mg | 1 mg | 1 mg |
| heptahydrate | | | | | |
| Ferrous sulfate | 5 mg | 5 mg | 50 mg | 5 mg | 10 mg |
| heptahydrate | | | | | |

TABLE 5
Composition of D-Tox YR culture medium (based on 1 L)

| | D-Tox | D-Tox | D-Tox | D-Tox |
|---|---|---|---|---|
| | Y_{0.1}^{0.5} R5 | Y₁^{0.5} R10 | Y₅^{0.5} R10 | Y₅⁰ R0 |
| Glucose | 5 g | 5 g | 5 g | 0 |
| Sodium nitrate | 3 g | 3 g | 3 g | 0 |
| Magnesium sulfate | 0.5 g | 0.5 g | 0.5 g | 0 |
| heptahydrate | | | | |
| Potassium chloride | 0.25 g | 0.25 g | 0.25 g | 0 |
| Potassium | 0.75 g | 0.75 g | 0.75 g | 0 |
| phosphate | | | | |
| Yeast extract | 0.1 g | 1 g | 5 g | 5 g |
| Ferrous sulfate | 5 mg | 10 mg | 10 mg | 0 |
| heptahydrate | | | | |

TABLE 6
Composition of D-Tox YR culture medium (based on 1 L)

| | D-Tox | D-Tox | D-Tox | D-Tox |
|---|---|---|---|---|
| | Y₅⁰ R10 | Y₅⁰ R20 | Y₅⁰ R40 | Y₅⁰ R80 |
| Glucose | 0 | 0 | 0 | 0 |
| Sodium nitrate | 0 | 0 | 0 | 0 |
| Magnesium sulfate | 0 | 0 | 0 | 0 |
| heptahydrate | | | | |
| Potassium chloride | 0 | 0 | 0 | 0 |
| Potassium phosphate | 0 | 0 | 0 | 0 |
| Yeast extract | 5 g | 5 g | 5 g | 5 g |
| Ferrous sulfate | 10 mg | 20 mg | 40 mg | 80 mg |
| heptahydrate | | | | |

### 3. Preparation of D-Tox

Aspergillus oryzae conidia were inoculated into a culture medium (1,000 ml) to have a final concentration of 5×10⁵ conidia/ml and incubated for 9 days at 30° C. with shaking at 150 rpm. Mycelia were separated from the culture broth by filtration through four layers of Miracloth (MilliporeSigma) and the sterile cell-free culture fermentate (D-Tox) was obtained by filtering through a 0.22 μm PES filter unit (Thermo Scientific, USA). It is preferable to keep D-Tox under refrigeration (4° C.), but it can be also kept within a broad temperature range like room temperature.

### 4. Preparation of Aflatoxin

Powder of AFB1 (aflatoxin B1) was purchased from Sigma Chemical Company, and standard solutions of AFB1 were prepared in acetonitrile according to the AOAC (Association of Official Analytical Chemists) method such that AFB1 has a final concentration of 10 μg/ml. Thus-prepared solutions were stored at −20° C. in amber glass vials.

### 5. Preparation of Patulin

Powder of patulin was purchased from Sigma Chemical Company, and standard solutions of patulin were prepared in ethyl acetate according to the AOAC method such that patulin has a final concentration of 10 μg/ml. Thus-prepared solutions were stored at −20° C. in amber glass vials.

### 6. Degradation of Aflatoxin B1 (AFB1) and Patulin by D-Tox

AFB1 or patulin (10 μl) was added at a concentration of 50 to 1,000 ppb (parts per billion) to D-Tox (20 ml). Then, after allowing the reaction to occur at various temperature and time conditions, degradation level of AFB1 and patulin was analyzed. As a control group of the culture filtrate, a culture filtrate which has not been inoculated with any Aspergillus was used. All the test groups and control groups were tested in a triplicate manner, and the degradation level of AFB1 and patulin was evaluated based on HPLC (high-performance liquid chromatography) analysis. AFB1 and patulin peak was recorded by using ChemStation software (Agilent, USA).

### 7. Extraction of AFB1 Using Organic Solvent

To extract AFB1 having hydrophobic property, chloroform was used for the pre-step of HPLC analysis as it is suitable for extracting non-polar and hydrophobic metabolites. For both the control and test groups, chloroform was added and mixed in an amount of 1 ml and 2 ml, respectively, and the reaction was allowed to occur. Thereafter, only the organic solvent portion was extracted and the organic solvent was evaporated in air. To the extracted AFB1, 1 ml of HPLC mobile phase (H₂O:CH₃OH:CH₃CN=50:40:10) was added to prepare a sample, which was then evaluated by HPLC analysis (Table 7).

### 8. Extraction of Patulin Using Organic Solvent

To extract patulin having water soluble property, ethyl acetate was used for the pre-step of HPLC analysis as it is suitable for extracting patulin. For both the control and test groups, ethyl acetate was added and mixed in an amount of 1 ml and 2 ml, respectively, and the reaction was allowed to occur. Thereafter, only the organic solvent portion was extracted and the organic solvent was evaporated in air. To the extracted patulin, 1 ml of HPLC mobile phase (1% acetic acid in H₂O:CH₃OH=95:5) was added to prepare a sample, which was then evaluated by HPLC analysis.

TABLE 7
Conditions for HPLC

| | |
|---|---|
| Equipment | Agilent 1100 HPLC system |
| | (degasser, autosampler, quaternary pump, coupled |
| | with a diode array detector, fluorescence detector) |
| Column | Zorbax Eclipse XDB-C18 4.6 mm X150 mm, 3.5 μm. |
| Detection | 365 nm for UV detection, |
| wavelength | 365 nm excitation and 450 nm emission for FLD |
| | detection |
| Mobile | For AFB1: H₂O:CH₃OH:CH₃CN = 50:40:10 |
| phase | For Patulin: 1% acetic acid in H₂O:CH₃OH = 95:5 |
| Flow rate | 0.8 ml/min |
| Injection | 10-100 μl |
| volume | |

It is also known that the lactone of AFB1 generally undergoes ring opening in strong basic state (pH 10 to 12), and, if the pH is lowered when no further degradation occurs, the lactone ring of AFB1 is restored to yield the toxin in original form. The inventors of the present invention named this phenomenon “reforming”, and, to examine any further degradation of AFB1 after the lactone ring opening by D-Tox, determination was made according to the following method. D-Tox sample was subjected to a reaction with AFB1 followed by heating for 60 minutes at 100° C., and, after 24 hours, the resulting sample was added with 6 N hydrochloric acid to adjust the pH to 2 to 3 and the reaction was allowed to occur for 4 hours at room temperature. After that, the reforming of AFB1 in original form with restored lactone ring was evaluated by HPLC analysis.

### Example 1. Determination of Suitability of Iron Sulfate Solution and Zinc Sulfate Solution for D-Tox Activity

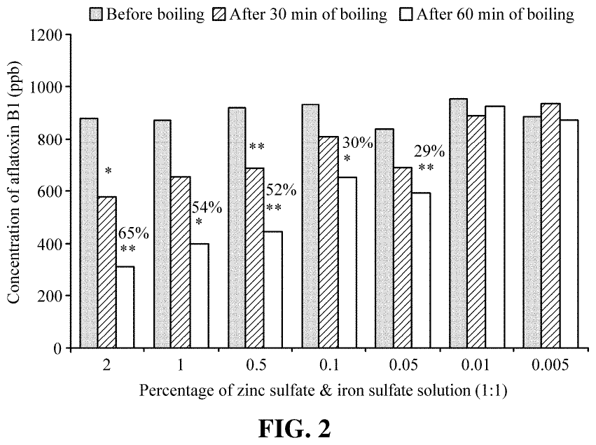

To determine whether or not an iron sulfate solution and a zinc sulfate solution exhibit a synergistic effect on the aflatoxin degrading activity of D-Tox, 1,000 ppb AFB1 was treated with iron sulfate or zinc sulfate over a broad concentration range and the reaction was allowed to occur for 60 minutes at 100° C. Then, the degradation level of AFB1 was analyzed. The inventors of the present invention prepared an iron sulfate solution and a zinc sulfate solution, each to have final concentration of 0.005, 0.01, 0.05, 0.1, 0.5, 1, or 2% (w/v), and mixed each solution at a ratio of 1:1 (50 ml:50 ml) to give a mixture solution. After that, the AFB1 degrading effect was determined over a broad concentration range (FIG. 2). As a result, it was found that, after heating for 60 minutes, AFB1 degrading activity of 65% is shown from 2% mixture solution of iron sulfate and zinc sulfate, but 0.005% solution and 0.01% solution did not show any difference in significant sense in terms of the AFB1 content even after heating for 60 minutes.

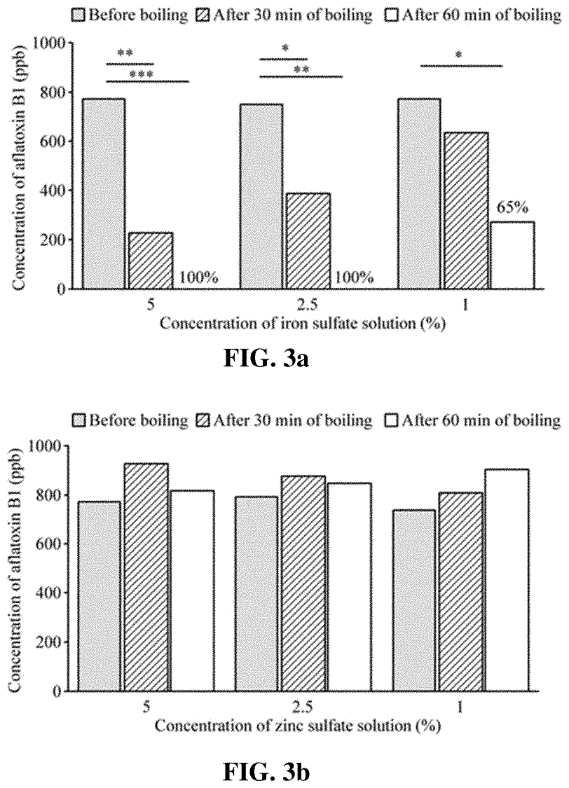

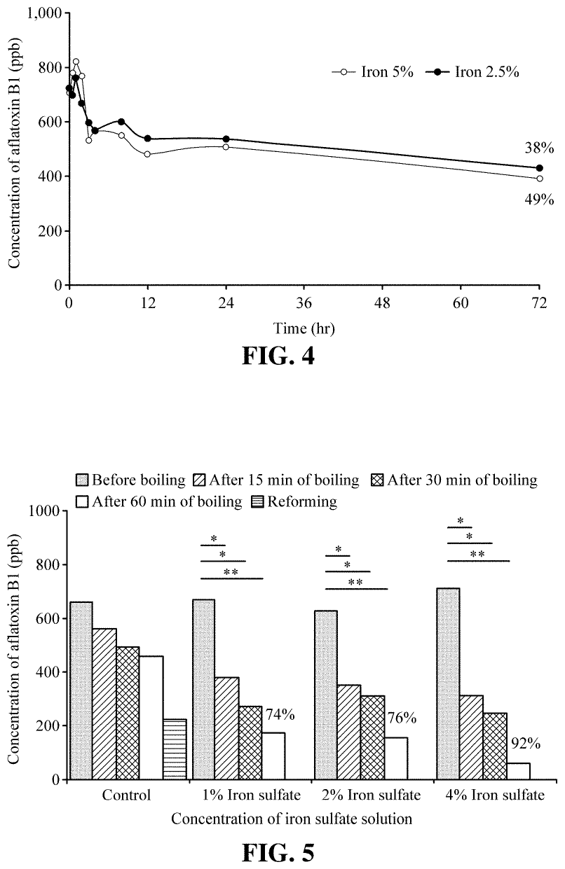

Furthermore, to examine the role played by each of iron sulfate and zinc sulfate, the inventors prepared each solution at 5, 2.5 and 1% (w/v) concentrations, and, after treating AFB1 with the solution at the same conditions as above to have a reaction, evaluated the degradation level of AFB1. As a result, 5% and 2.5% iron sulfate solutions showed AFB1 degrading activity of 100% after heating for 60 minutes as illustrated in FIG. 3a, but the zinc sulfate solution did not show any AFB1 degrading activity over the entire concentration range as illustrated in FIG. 3b. Based on these results, it is recognized that iron sulfate is a useful component for the AFB1 degrading activity of D-Tox, but zinc sulfate is a component which inhibits the AFB1 degrading activity of D-Tox. Nevertheless, since zinc sulfate has been known as a component promoting bacterial growth in culture medium containing Aspergillus, the inventors of the present invention tried to use zinc sulfate at low concentration. The corresponding results are shown in the following Example 3. When 1,000 ppb AFB1 was treated with 5% or 2.5% iron sulfate solution having high AFB1 degrading activity followed by storage at room temperature, AFB1 degrading activity of 49.5±7.7% and 38.1±3.9% was shown from each solution after 72 hours (FIG. 4). However, because a solution having high iron content may exhibit an adverse effect on the HPLC analysis, subsequent tests were carried out by an organic solvent extraction method using chloroform. As a result, 4% iron sulfate solution showed AFB1 degrading activity of 92% after heating for 60 minutes as illustrated in FIG. 5, but the control group showed AFB1 degrading activity of 31% after heating for 30 minutes, indicating that there is no difference in significant sense compared to the AFB1 content before the heating treatment. All the test groups were found to be in pH range of from 2.87 to 2.93, and therefore, as a control group of an iron sulfate solution, a solution prepared to have pH 3 at the same conditions as the test group by using 6N hydrochloric acid was used. Furthermore, as the pH after heating for 60 minutes was found to be 2.7 and 3.5 for the test groups and control group, respectively, reforming test was carried out only for the control group. Based on the obtained results, it was recognized that the reason of having irregular degradation performance between extraction of AFB1 with organic solvent (chloroform) and no extraction of AFB1 is caused by iron turbidity. It was also recognized that 4% iron sulfate solution having high AFB1 degrading activity is suitable for D-Tox.

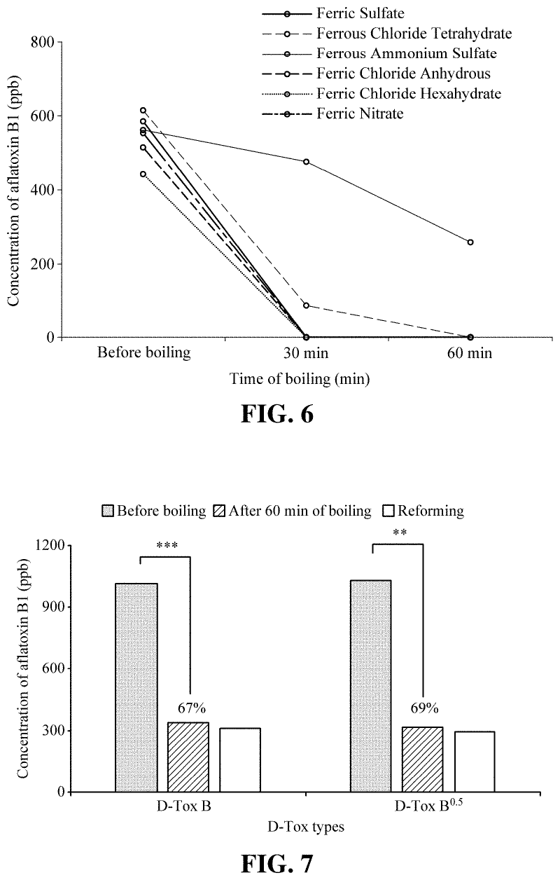

By assuming that the iron component included in 4% iron sulfate solution, which is suitable for D-Tox, is a D-Tox component, solutions having various iron components were prepared at 4% concentration. Iron components used for the preparation include ferric sulfate, ferric chloride anhydrous, ferrous chloride tetrahydrate, ferric chloride hexahydrate, ferrous ammonium sulfate, and ferric nitrate. The solutions prepared at 4% concentration were treated with 1,000 ppb AFB1, and, after heating for 60 minutes, analysis of AFB1 was carried out by an organic solvent extraction method using chloroform (FIG. 6). As a result, after heating for 60 minutes, 4% ferrous ammonium sulfate showed the AFB1 degrading activity of 53% while all other solutions showed the AFB1 degrading activity of 100%. In this regard, it was found that pH of 4% ferrous ammonium sulfate is 3.49, which is different from other solutions having lower pH (i.e., pH of about 1.57 to 2.60).

### Example 2. Determination of Aflatoxin Degrading Activity of D-Tox B at High Temperature (100° C.) Conditions

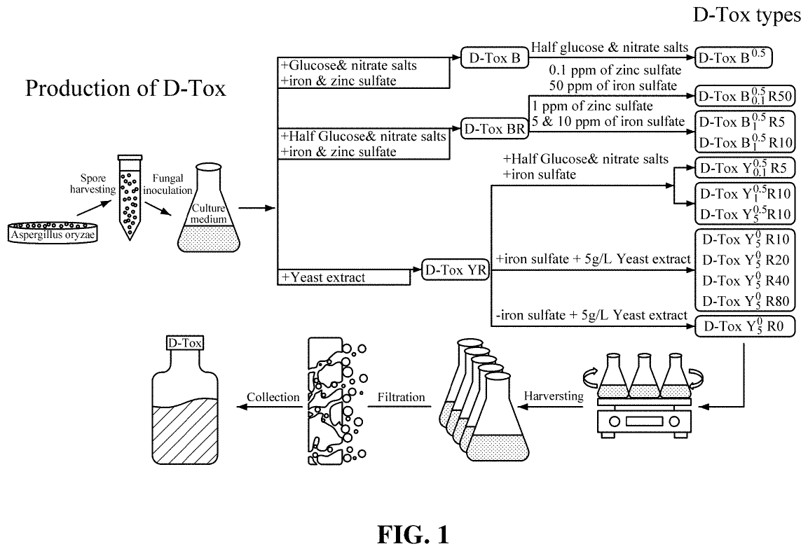

By culturing Aspergillus bacteria for 9 days in a culture medium consisting of glucose, sodium nitrate solution, zinc sulfate solution, and iron sulfate solution, the inventors of the present invention prepared D-Tox B (FIG. 1). AFB1 (1,000 ppb) was reacted with D-Tox B for 1 hour at 100° C., and then the degradation level and reforming of AFB1 were analyzed. In addition, to reduce the use amount of raw materials and to prepare D-Tox B in an environmentally friendly way, a culture medium in which content of glucose and sodium nitrate has been cut by half (½) of D-Tox B was used to prepare D-Tox B^{0.5}, which was then used for comparing the AFB1 degrading activity (see, Table 8). When both D-Tox B and D-Tox B^{0.5} are adjusted to pH 7.5, used for treatment with 1,000 ppb AFB1, and heated for 60 minutes, AFB1 degrading activity of 67% and 69% was obtained from each of them, respectively (FIG. 7). In addition, after heating for 60 minutes, pH was found to be 8.65 and 8.71 for D-Tox B and D-Tox B^{0.5}, respectively. As a result of culturing Aspergillus with composition D-Tox B^{0.5} and measuring the dry cell weight, the bacterial growth rate was found to be 0.14±0.02 mg, which is lower than the dry weight of D-Tox B, i.e., 0.33±0.04 mg. However, it was recognized that the AFB1 degrading activity is not different in significant sense compared to D-Tox B. There was also no difference in significant sense between the AFB1 content after heating for 60 minutes and AFB1 content after reforming test.

### Example 3. Optimization of D-Tox B^{0.5} Activity Based on Content of Zinc Sulfate and Iron Sulfate

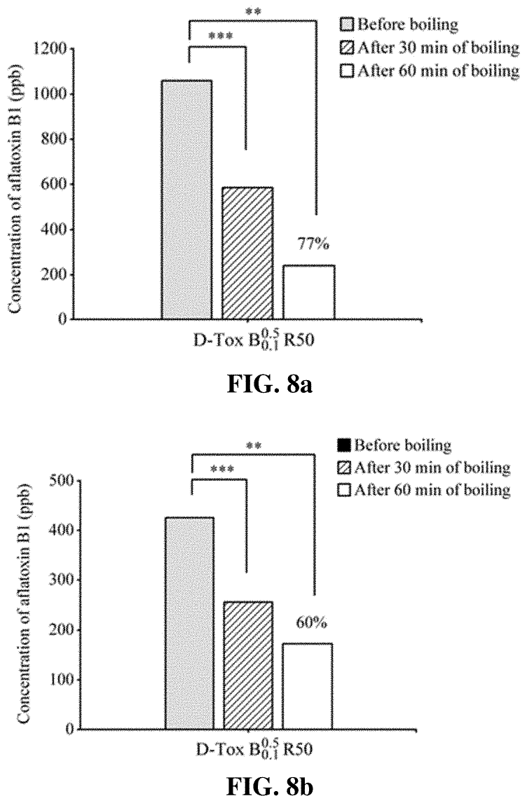

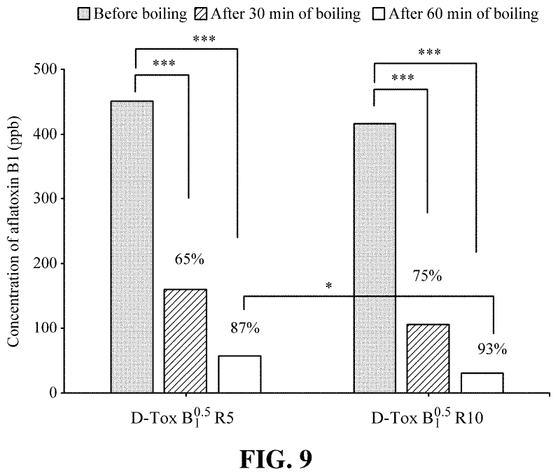

To optimize D-Tox B^{0.5} activity in Aspergillus culture broth, the inventors of the present invention carried out the tests by minimizing the use of zinc sulfate and increasing the content of iron sulfate according to Example 1. D-Tox B_{0.1}^{0.5} R50 was prepared such that zinc sulfate.heptahydrate and ferrous sulfate.heptahydrate have final concentration of 0.1 ppm and 50 ppm, respectively, per unit volume of the culture medium, and it was used for evaluating the AFB1 degrading activity (1,000 or 500 ppb AFB1) (FIGS. 8a-8b). After the reaction with AFB1 and heating at 100° C. for 30 minutes or 60 minutes, HPLC analysis was performed to evaluate the AFB1 degrading activity. As a result, D-Tox B_{0.1}^{0.5} R50 showed, after heating, the degrading activity of 77% for 1,000 ppb AFB1 as illustrated in FIG. 8a (FIG. 8a), while showing the degrading activity of 60% for 500 ppb AFB1 (FIG. 8b). Accordingly, it was recognized that zinc sulfate present at low concentration in culture medium slows down the growth rate of Aspergillus, and does not exhibit any constant degrading activity as shown in the result of FIGS. 8a-8b. Thus, the inventors of the present invention added zinc sulfate.heptahydrate such that it has final concentration of 1 ppm in unit volume of the culture medium and also added ferrous sulfate.heptahydrate such that it has final concentration of 5 ppm or 10 ppm in unit volume of the culture medium to newly prepare D-Tox B₁^{0.5} R5 and D-Tox B₁^{0.5} R10, which were then tested to compare their ability of degrading AFB1 (FIG. 9). D-Tox B₁^{0.5} R5, which has been prepared with the culture medium containing 5 ppm iron sulfate and 1 ppm zinc sulfate, exhibited the AFB1 degrading activity of 65% and 87%, respectively, after heating for 30 minutes or 60 minutes, thus indicating enhanced AFB1 degrading activity compared to D-Tox B^{0.5}. Furthermore, D-Tox B₁^{0.5} R10, which has been prepared with the culture medium containing 10 ppm iron sulfate and 1 ppm zinc sulfate (in final concentration), exhibited the AFB1 degrading activity of 75% and 93%, respectively, after heating for 30 minutes or 60 minutes. Thus, the AFB1 degrading activity was found to be higher in significant sense compared to D-Tox B₁^{0.5} R5 (p<0.05). It was shown based on these results that, compared to D-Tox B_{0.1}^{0.5} R50, D-Tox B₁^{0.5} R5 has a higher effect of enhancing the AFB1 degrading activity caused by controlling zinc sulfate, and the AFB1 degrading activity can be sufficiently and effectively enhanced by D-Tox B₁^{0.5} R10 rather than D-Tox B₁^{0.5} R5, i.e., by increasing the iron content.

### Example 4. Determination of Aflatoxin Degrading Activity of D-Tox B₁^{0.5} R10

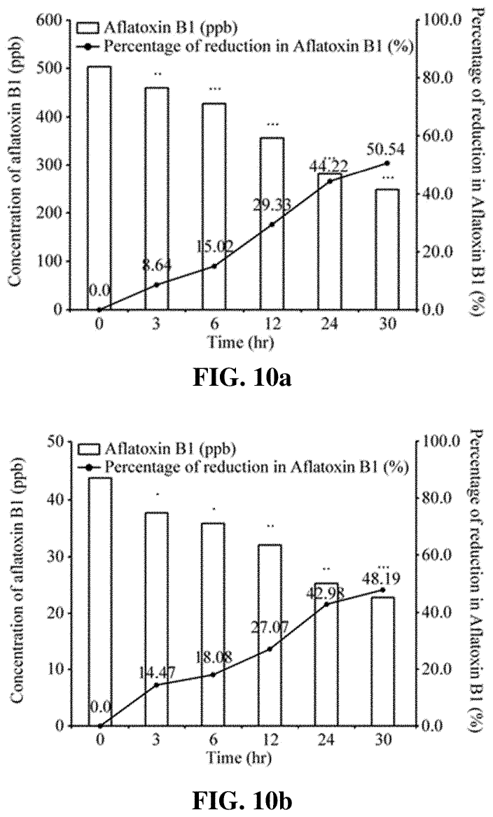

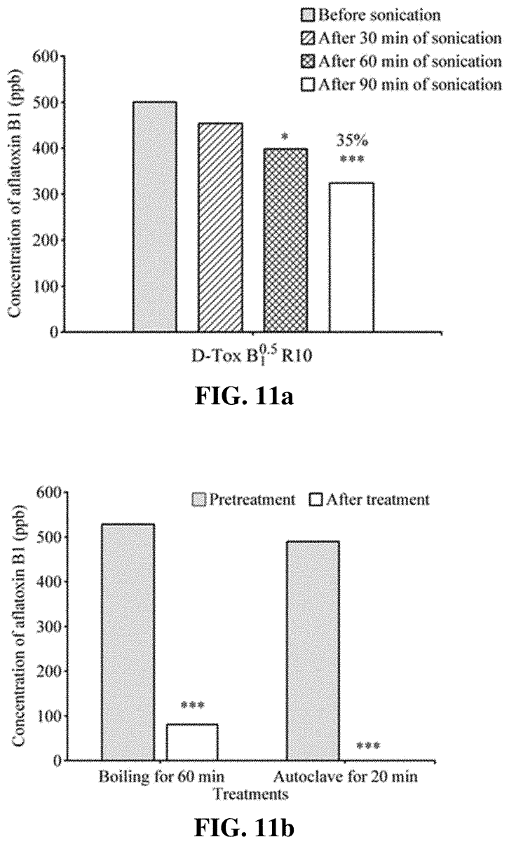

The inventors of the present invention determined the aflatoxin degrading activity of D-Tox B₁^{0.5} R10 not only at a condition of heating at high temperature (100° C.) but also after storage at room temperature, ultrasonication treatment (10-20 kHz), and high pressure sterilization (121° C., 20 minutes, 50 psi). First, D-Tox B₁^{0.5} R10 was reacted with AFB1 at 500 ppb and 50 ppb, respectively, for 30 hours at conditions including room temperature and 150 rpm shaking speed, and the degrading activity was evaluated by HPLC analysis. As a result, as it is shown in FIG. 10a, 503±18.4 ppb AFB1 turned into 249±7.1 ppb AFB1 after 30 hours at room temperature, representing the AFB1 degrading activity of 51%, and 44±0.1 ppb AFB1 turned into 23±0.9 ppb AFB1 after 30 hours at room temperature, representing the AFB1 degrading activity of 48% (FIG. 10b). Furthermore, D-Tox B₁^{0.5} R10 was added with AFB1 (500 ppb) and subjected to ultrasonication followed by keeping in a constant-temperature water bath for 30, 60, or 90 minutes. After that, amount of the remaining AFB1 was measured to obtain the degrading activity. As a result, it was found that, 30 minutes after the ultrasonication, AFB1 has remained with almost no degradation. However, the AFB1 degrading activity was 20% after 60 minutes following the ultrasonication, and it was 35% after 90 minutes following the ultrasonication (FIG. 11a). Still furthermore, when 500 ppb AFB1 was added to D-Tox B₁^{0.5} R10 followed by high pressure sterilization, a result showing 100% degradation of AFB1 was obtained (FIG. 11b). Based on these results, it was recognized that D-Tox B₁^{0.5} R10 of the present invention allows AFB1 degradation via various treatments, and higher AFB1 degrading activity is obtained from high temperatures.

### Example 5. Use Amount of Yeast Extract and Optimization of D-Tox Activity

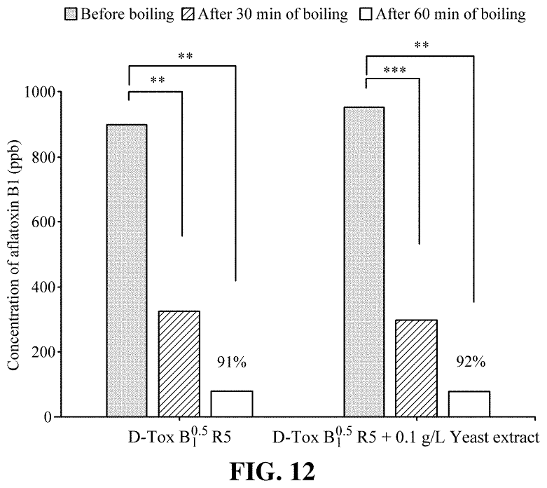

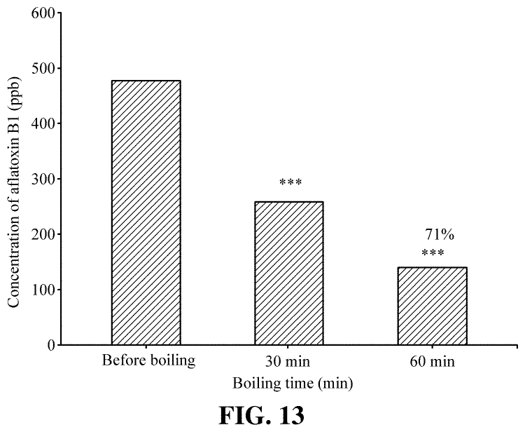

To determine the efficiency of AFB1 degrading activity by the yeast extract in Aspergillus culture broth, yeast extract (0.1 g/L) was added to D-Tox B₁^{0.5} R5 and culture was performed for 9 days to carry out a series of gain-of-function test. Specifically, after reaction with AFB1 and heating for 30 minutes or 60 minutes at 100° C., the AFB1 degrading activity was evaluated by HPLC analysis. As a result, as it is illustrated in FIG. 12, both the D-Tox culture broth to which 0.1 g/L yeast extract has been added and D-Tox culture broth to which no yeast extract has been added exhibited the degrading activity of 91-92% for 1,000 ppb AFB1. Inventors of the present invention prepared culture broth of D-Tox Y_{0.1}^{0.5} R5, which has been prepared by adding 0.1 g/L yeast extract instead of the trace element, i.e., zinc sulfate, in D-Tox B₁^{0.5} R5, and used it for a reaction with 500 ppb AFB1 to analyze the degradation level (FIG. 13). As a result of analyzing the degrading activity by measuring the residual amount of AFB1, the AFB1 degrading activity of 71% was shown after heating for 60 minutes. Based on this result, it was realized that yeast extract can be used instead of zinc sulfate, which is an essential element for bacterial growth in Aspergillus culture broth, and yeast extract enables degradation of AFB1.

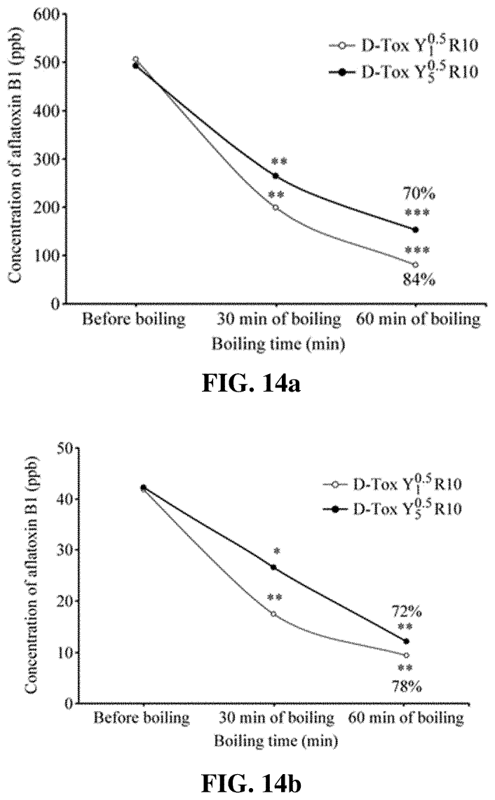

To optimize D-Tox activity based on the above result, yeast extract and iron sulfate were additionally added to the culture medium to prepare D-Tox YR series, and then the AFB1 degrading activity was compared among the series. D-Tox Y₁^{0.5} R10 and D-Tox Y₅^{0.5} R10 represent a culture filtrate in which glucose and sodium nitrate are added such that they are ½ of D-Tox B, ferrous sulfate.heptahydrate is added to have final concentration of 10 pm per unit volume of the culture medium, and yeast extract is added to the culture medium in an amount of 1 g/L and 5 g/L, respectively. D-Tox YR series was reacted with 500 ppb or 50 ppb AFB1, heated at 100° C. for 30 minutes or 60 minutes, and then subjected to HPLC analysis to evaluate the degradation level of AFB1. As a result, D-Tox Y₅^{0.5} R10, which has been prepared with a culture medium containing 5 g/L yeast extract, showed the AFB1 degrading activity of 69% after heating for 60 minutes, i.e., from 492±7.1 ppb to 152±11.9 ppb AFB1, while D-Tox Y₁^{0.5} R10, which has been prepared with a culture medium containing 1 g/L yeast extract, showed the AFB1 degrading activity of 84% after heating for 60 minutes, i.e., from 507±22.7 ppb to 80±6.8 ppb AFB1 (FIG. 14a). Furthermore, when reacted with 50 ppb AFB1 followed by heating for 60 minutes at 100° C., D-Tox Y₅^{0.5} R10 showed AFB1 degrading activity of 72%, i.e., from 42±2.3 ppb to 12±0.3 ppb AFB1 while D-Tox Y₁^{0.5} R10 showed AFB1 degrading activity of 78%, i.e., from 42±1.4 ppb to 9±0.7 ppb AFB1 (FIG. 14b).

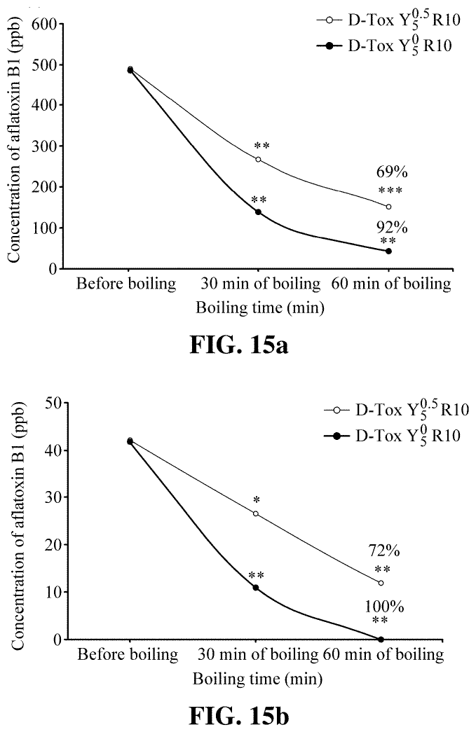

In addition, the AFB1 degrading activity of D-Tox Y₅⁰ R10, which has been prepared by removing glucose and sodium nitrate from the culture medium, was determined. The culture filtrate D-Tox Y₅⁰ R10 was prepared by adding ferrous sulfate.heptahydrate such that it has final concentration of 10 ppm per unit volume of the culture medium and also adding 5 g/L yeast extract to the culture medium. D-Tox Y₅⁰ R10 and D-Tox Y₅^{0.5} R10 were reacted with 500 ppb or 50 ppb AFB1, heated at 100° C. for 30 minutes or 60 minutes, and then subjected to HPLC analysis to evaluate the AFB1 degrading activity. As a result, as it is illustrated in FIG. 15a, D-Tox Y₅⁰ R10, which has been prepared with a culture medium with removal of glucose and sodium nitrate, showed the AFB1 degrading activity of 92%, i.e., from 488±26.6 ppb to 41±1.1 ppb AFB1. Furthermore, D-Tox Y₅^{0.5} R10 prepared by reaction with 50 ppb AFB1 and heating at 100° C. for 60 minutes showed the AFB1 degrading activity of 72%, while D-Tox Y₅⁰ R10 showed the AFB1 degrading activity of 100% (FIG. 15b). In particular, as a result of measuring the pH of reaction solution of all test groups after heating for 60 minutes, it was found that there is an increase in pH, i.e., from 7.55 to 8.51 for D-Tox Y_{0.1}^{0.5} R5, from 8.26 to 8.93 for D-Tox Y₁^{0.5} R10, and from 7.58 to 8.81 for D-Tox Y₅^{0.5} R10. In this regard, a huge increase in pH, i.e., from 8.36 before the reaction to 9.21, was shown from D-Tox Y₅⁰ R10 test group which exhibits the excellent AFB1 degrading activity. Based on these results, it was recognized that D-Tox Y₅⁰ R10 has better AFB1 degrading activity than D-Tox Y₅^{0.5} R10, and, in case of reducing the content of yeast extract in D-Tox Y₅^{0.5} R10, the AFB1 degrading activity can be effectively enhanced just with 1 g/L yeast extract (i.e., D-Tox Y₁^{0.5} R10).

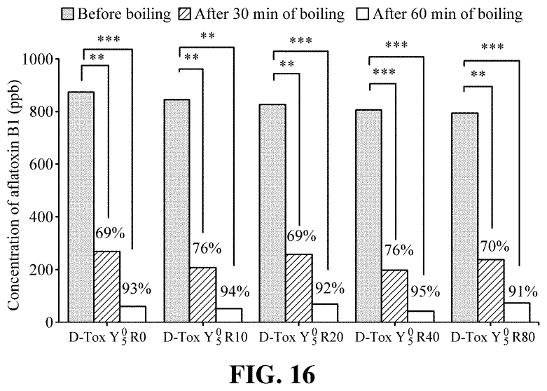

To optimize D-Tox Y₅⁰ R activity according to iron sulfate content based on the above result, culture medium was prepared with varying iron sulfate concentration and the AFB1 degrading activity was compared among the different concentrations. Specifically, D-Tox Y₅⁰ R0, D-Tox Y₅⁰ R10, D-Tox Y₅⁰ R20, D-Tox Y₅⁰ R40, and D-Tox Y₅⁰ R80 mean a culture filtrate which has been prepared by adding, to culture medium, ferrous sulfate.heptahydrate to have final concentration of 0, 10, 20, 40, and 80 ppm, respectively, per unit volume of the culture medium and adding, to a culture medium, yeast extract in an amount of 5 g/L. The above D-Tox Y₅⁰ R_{S} were reacted with 1,000 ppb AFB1, heated at 100° C. for 30 minutes or 60 minutes, and then subjected to HPLC analysis to evaluate the AFB1 degrading activity. As a result, the AFB1 degrading activity of 91-95% was shown from all of them after heating for 60 minutes, regardless of the content of iron sulfate as it is illustrated in FIG. 16. Furthermore, as the pH has increased to pH 9 or higher from pH 8.7 before heating, it was found to be useful for enhancing the efficiency of AFB1 degrading activity.

### Example 6. Determination of Patulin Degrading Activity by D-Tox BR and D-Tox YR

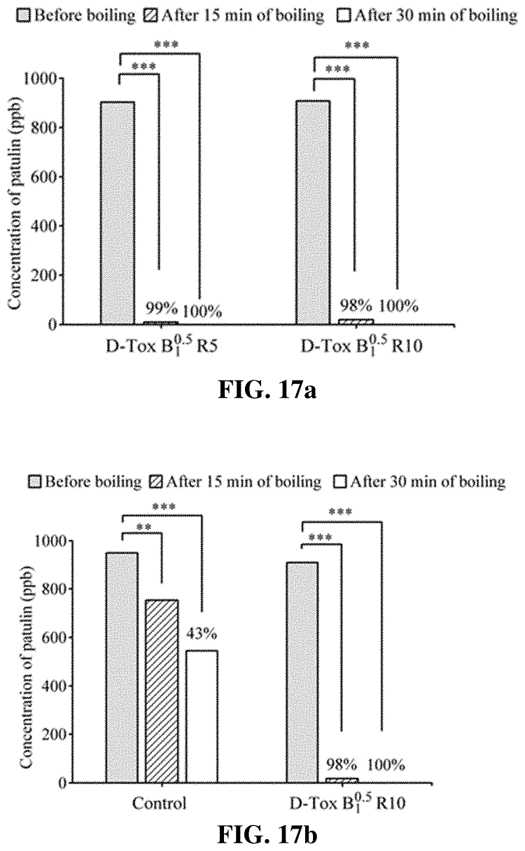

The inventors of the present invention carried out a test to see whether or not various kinds of D-Tox BR and D-Tox YR described above are suitable for degrading patulin as well as aflatoxin at high temperature conditions (i.e., by HPLC analysis after heating for 15 minutes or 30 minutes at 100° C.). From D-Tox BR series, D-Tox B₁^{0.5} R5 and D-Tox B₁^{0.5} R10 which have been found to have very high aflatoxin degrading activity were selected, and both exhibited high patulin degrading activity of 98-99% after heating for 15 minutes, and, after heating for 30 minutes, the patulin degrading activity of 100% was shown (FIG. 17a). Furthermore, when comparison is made between the control group, which is a culture filtrate not inoculated with any Aspergillus, and D-Tox B₁^{0.5} R10, a difference in significant sense was shown. From the control group, which is a culture filtrate not inoculated with any Aspergillus, patulin degrading activity of 43%, i.e., from 950±25.4 ppb to 546±9.9 ppb patulin, was shown after heating for 30 minutes while D-Tox B₁^{0.5} R10 showed the 100% patulin degrading activity, i.e., from 909±20.2 ppb to 0 ppb patulin (FIG. 17b).

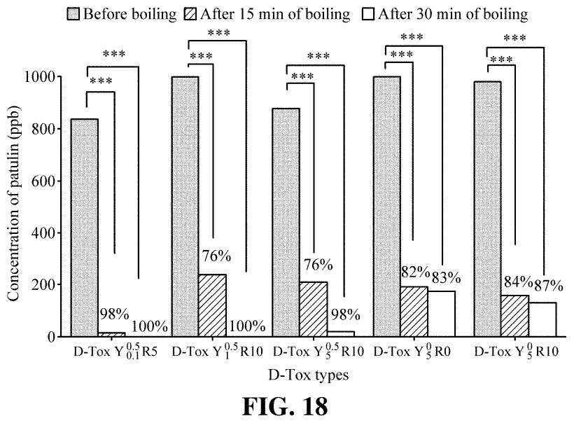

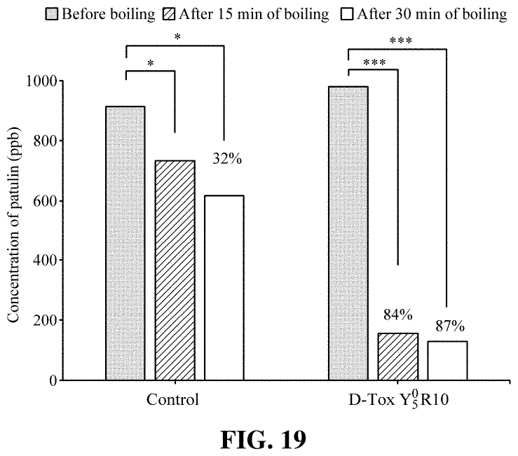

To determine the efficiency of patulin degrading activity by yeast extract in the culture broth of Aspergillus, the entire D-Tox YR series was reacted with 1,000 ppb patulin, heated at 100° C. for 15 minutes or 30 minutes, and then subjected to HPLC analysis to evaluate the patulin degrading activity. As a result, as it is illustrated in FIG. 18, D-Tox Y_{0.1}^{0.5} R5, i.e., a culture medium containing 0.1 g/L yeast extract, showed the patulin degrading activity of 98%, i.e., from 839±14.7 ppb to 13±1.7 ppb patulin after heating for 15 minutes. In case of heating for 30 minutes, D-Tox Y_{0.1}^{0.5} R5 showed the patulin degrading activity of 100%. In addition, in case of D-Tox Y₁^{0.5} R10 and D-Tox Y₅^{0.5} R10, which were prepared with a culture medium to which 1 g/L or 5 g/L yeast extract has been added, the patulin degrading activity of 76% was shown from both after heating for 15 minutes, while patulin degrading activity of 100% and 98% was shown from each of them, respectively, after hating for 30 minutes. On the other hand, in case of D-Tox Y₅⁰ R0 and D-Tox Y₅⁰ R10 which are a culture medium prepared by removing glucose and sodium nitrate, patulin degrading activity of 83%, i.e., from 1063±37.2 ppb to 175±8.3 ppb patulin after heating for 30 minutes, and patulin degrading activity of 87%, i.e., from 980±25.0 ppb to 130±8.7 ppb patulin after heating for 30 minutes were shown, respectively. When the control group is compared with D-Tox Y₅⁰ R10, D-Tox Y₅⁰ R10 showed a significantly higher patulin degrading activity (i.e., 87%) compared to the control group which showed the patulin degrading activity of 32% after heating for 30 minutes. Based on this result, it was shown that the culture broth added with Aspergillus strain has indeed brought higher efficiency of D-Tox activity (FIG. 19). In particular, as a result of measuring pH of the reaction solution of every test group after heating for 30 minutes, an increase in pH was shown after the heating, i.e., from 7.86 to 8.11 for D-Tox Y_{0.1}^{0.5} R5, from 7.04 to 7.58 for D-Tox Y₁^{0.5} R10, from 7.53 to 8.45 for D-Tox Y₅^{0.5} R10, from 8.83 to 9.12 for D-Tox Y₅⁰ R0, and from 8.77 to 9.15 for D-Tox Y₅⁰ R10. Moreover, it was found from D-Tox Y₁^{0.5} R10 showing higher patulin degrading activity but with lower pH than other D-Tox YR after heating that the patulin degrading activity is less affected by pH compared to the aflatoxin degrading activity. It was also found from the above result that, unlike the aflatoxin degrading activity, only a small amount of yeast extract required for bacterial growth is effective for the patulin degrading activity, and yeast extract itself can increase the efficiency of patulin degrading activity regardless of the content of iron sulfate.

TABLE 8
AFB1 degrading activity for different types of D-
Tox after heating for 60 minutes at 100° C.

| | | AFB1-degradating activity | |
|---|---|---|---|
| Type of D-Tox | Name of D-Tox | (%) | Reform (%) |
| D-Tox B | D-Tox B | 66.6 ± 1.56 | 0 |
| | D-Tox B^{0.5} | 69.1 ± 2.59 | 0 |
| D-Tox BR | D-Tox B_{0.1}^{0.5} R50 | 59.8-77.5 | ND |
| | D-Tox B₁^{0.5} R5 | 87.3 ± 1.45 | ND |
| | D-Tox B₁^{0.5} R10 | 92.5 ± 0.76 | ND |
| D-Tox YR | D-Tox y_{0.1}^{0.5} R5 | 70.8 ± 0.80 | ND |
| | D-Tox Y₁^{0.5} R10 | 78.0-84.2 | ND |
| | D-Tox Y₅^{0.5} R10 | 69.2-71.7 | ND |
| | D-Tox Y₅⁰ R10 | 91.5-100 | ND |
| | D-Tox Y₅⁰ R0 | 93.1 ± 1.52 | ND |
| | D-Tox Y₅⁰ R20 | 91.6 ± 0.26 | ND |
| | D-Tox Y₅⁰ R40 | 94.6 ± 1.81 | ND |
| | D-Tox Y₅⁰ R80 | 90.7 ± 0.87 | ND |

(ND: Not Determined)

TABLE 9
Patulin degrading activity for different types of D-Tox after
heating for 30 minutes at 100° C.

| | | Patulin degrading activity |
|---|---|---|
| Type of D-Tox | Name of D-Tox | (%) |
| D-Tox BR | D-Tox B₁^{0.5} R5 | 100 |
| | D-Tox B₁^{0.5} R10 | 100 |
| D-Tox YR | D-Tox Y_{0.1}^{0.5} R5 | 100 |
| | D-Tox Y₁^{0.5} R10 | 100 |
| | D-Tox Y₅^{0.5} R10 | 83.5 ± 1.25 |
| | D-Tox Y₅⁰ R10 | 86.7 ± 0.94 |

1. A composition for degradation of mycotoxin comprising, as an effective component, (a) an iron compound; (b) yeast extract; or (c) culture filtrate of Aspergillus cultured in culture medium containing one or more of an iron component and yeast extract.

2. The composition for degradation of mycotoxin according to claim 1, wherein the iron compound is an iron salt.

3. The composition for degradation of mycotoxin according to claim 1, wherein the Aspergillus is Aspergillus oryzae, Aspergillus terreus, Aspergillus sojae, Aspergillus nidulans, Aspergillus fumigatus, or Aspergillus flavus.

4. The composition for degradation of mycotoxin according to claim 1, wherein the mycotoxin is aflatoxin, patulin, or ochratoxin.

5. A method for degradation of mycotoxin including treating a sample suspected to contain mycotoxin with the composition of claim 1.

6. The method for degradation of mycotoxin according to claim 5, wherein the sample suspected to contain mycotoxin is an agricultural product, a processed food product, or an animal feed.

7. The method for degradation of mycotoxin according to claim 5, wherein the mycotoxin is aflatoxin, patulin, or ochratoxin.

8. A method for production of Aspergillus culture filtrate having an activity of degrading mycotoxin including:
(a) inoculating Aspergillus conidia to a culture medium containing one or more of an iron compound and yeast extract followed by culturing; and
(b) filtering a culture broth of the Aspergillus of above (a).

9. The method for production of Aspergillus culture filtrate having an activity of degrading mycotoxin according to claim 8, wherein the Aspergillus is Aspergillus oryzae, Aspergillus terreus, Aspergillus sojae, Aspergillus nidulans, Aspergillus fumigatus, or Aspergillus flavus.

10. The method for production of Aspergillus culture filtrate having an activity of degrading mycotoxin according to claim 8, wherein the culture medium contains 0.1-300 mg of iron compound per 1 L unit volume of the culture medium.

11. The method for production of Aspergillus culture filtrate having an activity of degrading mycotoxin according to claim 8, wherein the culture medium contains 0.01-10 g of yeast extract per 1 L unit volume of the culture medium.

12. The method for production of Aspergillus culture filtrate having an activity of degrading mycotoxin according to claim 8, wherein the mycotoxin is aflatoxin, patulin, or ochratoxin.

13. An Aspergillus culture filtrate having an activity of degrading mycotoxin that is produced by the method of claim 8.

14. A food additive comprising the composition for degradation of mycotoxin of claim 1.

15. An animal feed additive comprising the composition for degradation of mycotoxin of claim 1.
